(19) United States
(12) Patent Application Publication
(10) Pub. No.: US 2025/0229293 A1
(43) Pub. Date: Jul. 17, 2025

(54) ULTRASOUND PROBE

(71) Applicant: FUJIFILM Corporation, Tokyo (JP)

(72) Inventors: Hideki OKAZAKI, Tokyo (JP); Kazuho YOSHIMURA, Tokyo (JP)

(73) Assignee: FUJIFILM Corporation, Tokyo (JP)

(21) Appl. No.: 19/008,537

(22) Filed: Jan. 2, 2025

(30) Foreign Application Priority Data
Jan. 16, 2024 (JP) 2024-004384

(51) Int. Cl. B06B 1/06; H10N 30/87

(52) U.S. Cl. CPC B06B 1/0629; H10N 30/875

(57) ABSTRACT

A transducer array has two dummy transducers adjacent to an effective part. Each dummy transducer has a conductive part consisting of a dummy piezoelectric layer and a dummy reflective layer. A ground line in a wiring sheet is electrically connected to a ground film through two conductive parts. Two second slits are provided between the two dummy transducers and the effective part. The wiring sheet has a lower wiring pattern, an upper wiring pattern, and a via group.

### CROSS-REFERENCE TO RELATED APPLICATIONS

This application claims benefit of priority to Japanese Patent Application No. 2024-004384 filed Jan. 16, 2024, the entire contents of which are incorporated herein by reference.

### BACKGROUND OF THE INVENTION

### 1. Field of the Invention

The present disclosure relates to an ultrasound probe, and more particularly to a structure of the ultrasound probe.

### 2. Description of the Related Art

In an ultrasound examination, an ultrasound probe is used. An ultrasound probe which is in contact with a body surface of a subject, an ultrasound probe which is inserted into the subject, an ultrasound probe which is incorporated in an endoscope, and the like are known. The ultrasound probe incorporated in the endoscope may also be referred to as an ultrasound sensor.

The ultrasound probe includes an assembly disposed in a case. The assembly has, for example, a backing, a wiring sheet, a transducer array, a ground film, and an acoustic lens. The transducer array is composed of a plurality of transducers arranged in a longitudinal direction. The longitudinal direction is a transducer arrangement direction, which is also referred to as a major axis direction. A direction orthogonal to the longitudinal direction is referred to as a lateral direction or a minor axis direction. The wiring sheet is generally formed of a flexible printed circuit (FPC) substrate. The ground film is formed of a copper foil and the like.

The plurality of transducers constituting the transducer array each have a piezoelectric layer. Each piezoelectric layer is formed of a piezoelectric material, which is a piezoelectric electro-mechanical conversion material, and has a signal electrode and a ground electrode. A plurality of signal electrodes of a plurality of piezoelectric layers are connected to a plurality of signal lines in the wiring sheet. A plurality of ground electrodes of the plurality of piezoelectric layers are connected to one or a plurality of ground lines in the wiring sheet through the ground film.

FIG. 2 of JP2013-150681A discloses an ultrasound probe having a transducer array and a wiring sheet. The wiring sheet has a first part provided on a lower side of the transducer array, and a pair of second parts pulled out from both sides of the first part in a longitudinal direction. FIG. 9 of JP2013-150681A shows a structure for connecting a signal line and a ground line to a piezoelectric layer.

FIGS. 3 and 7 of WO2020/079855A also disclose a transducer array and a wiring sheet. JP2013-150681A and WO2020/079855A do not disclose a dummy transducer for ground wiring.

### SUMMARY OF THE INVENTION

In the assembly disposed in the ultrasound probe, in a case in which both end parts of the ground film are connected to the ground line of the wiring sheet by soldering, there is a problem in that the workload is increased, it is necessary to reserve a region for soldering on the wiring sheet, or thermal damage to a piezoelectric layer array.

An object of the present disclosure is to enable easy and reliable ground wiring in an ultrasound probe. Alternatively, an object of the present disclosure is to provide a transducer array having good electrical characteristics and good physical characteristics.

The present disclosure relates to an ultrasound probe comprising: a transducer array that has a plurality of real transducers having a plurality of real piezoelectric layers and a dummy transducer having a dummy piezoelectric layer; a ground film that is provided on an upper side of the plurality of real piezoelectric layers and the dummy piezoelectric layer and that is electrically connected to a plurality of ground electrodes of the plurality of real piezoelectric layers and an upper surface electrode of the dummy piezoelectric layer; and a wiring sheet that is provided on a lower side of the transducer array and that has a plurality of signal lines electrically connected to a plurality of signal electrodes of the plurality of real piezoelectric layers and a ground line electrically connected to the ground film, in which the dummy transducer has a conductive part that is provided between the ground film and the wiring sheet and that includes the dummy piezoelectric layer, and the ground film and the ground line are electrically connected to each other through the conductive part.

According to the present disclosure, in the ultrasound probe, it is possible to easily and reliably perform the ground wiring. Alternatively, according to the present disclosure, it is possible to provide the transducer array having good electrical characteristics and good physical characteristics.

### BRIEF DESCRIPTION OF THE DRAWINGS

FIG. 1 is a cross-sectional view showing an ultrasound probe according to an embodiment.

FIG. 2 is a cross-sectional view showing an assembly.

FIG. 3 is a plan view showing a wiring sheet.

FIG. 4 is a view showing an upper wiring pattern and a lower wiring pattern.

FIG. 5 is a perspective view showing a part of an intermediate assembly.

FIG. 6 is a developed view showing an example of a ground film.

FIG. 7 is a view showing a first example of a wiring pattern.

FIG. 8 is a view showing a second example of the wiring pattern.

FIG. 9 is a view showing a third example of the wiring pattern.

FIG. 10 is a flowchart showing a method of manufacturing the ultrasound probe according to the embodiment.

### DESCRIPTION OF THE PREFERRED EMBODIMENTS

Hereinafter, an embodiment will be described based on the accompanying drawings.

### (1) Summary of Embodiment

An ultrasound probe according to the embodiment has a transducer array, a ground film, and a wiring sheet. The transducer array has a plurality of real transducers having a plurality of real piezoelectric layers, and a dummy transducer having a dummy piezoelectric layer. The ground film is provided on an upper side of the plurality of real piezoelectric layers and the dummy piezoelectric layer. More specifically, the ground film is electrically connected to a plurality of ground electrodes of the plurality of real piezoelectric layers and an upper surface electrode of the dummy piezoelectric layer. The wiring sheet is provided on a lower side of the transducer array. More specifically, the wiring sheet has a plurality of signal lines that are electrically connected to a plurality of signal electrodes of the plurality of real piezoelectric layers, and a ground line that is electrically connected to the ground film. The dummy transducer has a conductive part provided between the ground film and the wiring sheet. The conductive part includes the dummy piezoelectric layer. The ground film and the ground line are electrically connected to each other through the conductive part.

With the above-described configuration, since the ground film and the ground line are electrically connected to each other through the conductive part in the dummy transducer, it is not necessary to solder the ground film to the wiring sheet. Therefore, it is possible to easily and reliably perform the ground wiring. In addition, it is possible to maintain or improve the acoustic characteristics of the transducer array. In the present specification, the expression of electrical connection may include direct connection and indirect connection.

In the embodiment, the dummy piezoelectric layer has an upper surface electrode, a lower surface electrode, and a side surface electrode connected to the upper surface electrode and the lower surface electrode. The side surface electrode functions as a short-circuit electrode. Two dummy transducers may be provided on both sides of an effective part including the plurality of real transducers. Three or more dummy transducers may be provided. The side surface electrode is an electrode formed on a vertical surface (surface intersecting a transducer arrangement direction) in the piezoelectric layer.

In the embodiment, the plurality of real transducers have a plurality of real reflective layers that are provided on a lower side of the plurality of real piezoelectric layers and that have conductivity. The conductive part has a dummy reflective layer that is provided on a lower side of the dummy transducer and that has conductivity. Each of the real reflective layers has a function of reflecting ultrasound emitted from the corresponding piezoelectric layer toward a lower side. In the dummy transducer, the dummy piezoelectric layer and the dummy reflective layer both function as electrical connection components.

In the embodiment, the transducer array has a longitudinal direction. A width of the dummy transducer in the longitudinal direction is larger than a width of each real transducer in the longitudinal direction. With this configuration, an electric resistance of a ground path is reduced. Further, it is possible to physically reinforce the transducer array. In general, at both end parts of the transducer array in the longitudinal direction, transducer damage (specifically, transducer collapse) is likely to occur. With the above-described configuration, such a problem is less likely to occur.

In the embodiment, the transducer array has the effective part and a second slit. The effective part is composed of the plurality of real transducers and a plurality of first slits for separating the respective real transducers from each other. The second slit is provided between the effective part and the dummy transducer. A width of the second slit in the longitudinal direction is larger than a width of each of the first slits in the longitudinal direction.

With the above-described configuration, the insulating properties can be improved between the real transducer present at the end part of the effective part and the dummy transducer adjacent thereto. For example, in a case in which the effective part is subjected to repolarization processing, a short circuit between the real transducer present at the end part and the dummy transducer adjacent thereto is less likely to occur. In a case in which the transducer array has a curved form, the width of the second slit in the longitudinal direction is a width of a bottom part of the second slit, and similarly, the width of each first slit in the longitudinal direction is a width of a bottom part of each first slit.

In the embodiment, the ground film includes a film body having a plurality of strips, and a pair of film end parts that are connected to the plurality of strips. The plurality of strips consist of a plurality of strips electrically bonded to the plurality of ground electrodes and a strip electrically bonded to the upper surface electrode of the dummy piezoelectric layer. Each strip is an elongated piece or a strip-shaped part.

In the embodiment, the transducer array has a longitudinal direction and a lateral direction intersecting the longitudinal direction. The wiring sheet has an upper wiring pattern, a lower wiring pattern, and a via group. The upper wiring pattern has a plurality of upper signal lines that are electrically connected to a plurality of signal electrodes of the plurality of real transducers and that are arranged in the longitudinal direction, and an upper ground line that is electrically connected to a lower surface electrode of the dummy transducer. The lower wiring pattern has a plurality of lower signal lines arranged in the lateral direction and a lower ground line. The via group has a plurality of signal vias that connect the plurality of upper signal lines and the plurality of lower signal lines to each other, and a ground via that electrically connects the upper ground line and the lower ground line to each other.

In the embodiment, the wiring sheet has a sheet body, a first stretching part, and a second stretching part. The sheet body is provided on a lower side of the transducer array. The sheet body has one end and the other end that are separated from each other in the longitudinal direction. The first stretching part is a part that is pulled out from the one end of the sheet body. The second stretching part is a part pulled out from the other end of the sheet body. The plurality of real transducers are n real transducers from a first real transducer to an n-th real transducer that are arranged in order of numbers in the longitudinal direction. The plurality of lower signal lines include a first lower signal line group and a second lower signal line group. The first lower signal line group is electrically connected to odd-numbered real transducers among the n real transducers, and is provided over the sheet body and the first stretching part. The second lower signal line group is electrically connected to an even-numbered real transducer among the n real transducers, and is provided over the sheet body and the second stretching part.

In the embodiment, the plurality of signal vias include a first signal via row that is connected to the first lower signal line group and a second signal via row that is connected to the second lower signal line group. The first signal via row is aligned along a diagonal direction of the sheet body. The second signal via row is aligned along the diagonal direction while being shifted from the first signal via row.

With the above-described configuration, it is possible to reduce an inclination angle of each signal via row with respect to the longitudinal direction while ensuring a certain inter-signal via pitch. Therefore, a width of the wiring sheet in the lateral direction can be reduced. The ultrasound probe according to the embodiment includes a first assembly (intermediate assembly), a backing, and a case. The first assembly consists of the transducer array, the ground film, and the wiring sheet. The backing is provided on a lower side of the first assembly. The backing has a first end part and a second end part which are separated from each other in the longitudinal direction. The case accommodates a second assembly consisting of the first assembly and the backing. The wiring sheet has the sheet body, the first stretching part, and the second stretching part. The sheet body is a part provided on the lower side of the transducer array. The sheet body has one end and the other end that are separated from each other in the longitudinal direction. The first stretching part is a part that is pulled out from the one end of the sheet body. The second stretching part is a part pulled out from the other end of the sheet body. The first stretching part enters a lower space through a space adjacent to the first end part and then makes a U-shaped turn in the lower space. The second stretching part enters the lower space through a space adjacent to the second end part. An end part of the first stretching part and an end part of the second stretching part overlap each other on the lower side of the backing.

With the above-described configuration, since the two end parts of the two stretching parts overlap each other, even in a case in which the lower space is a small space, it is possible to connect a large number of cables (for example, 100 or more cables) to the wiring sheet by using the lower space.

### (2) Details of Embodiment

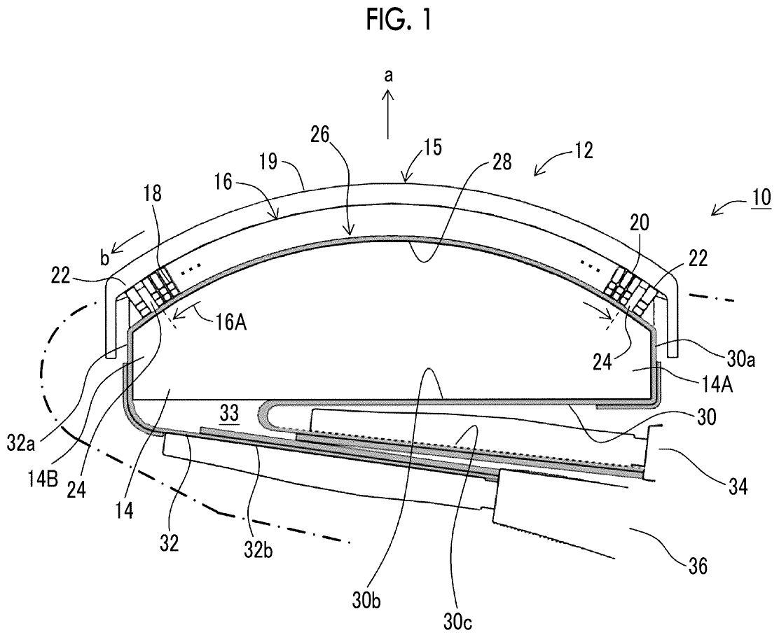

FIG. 1 shows a distal end part 10 of an endoscope. The endoscope includes an imaging element, an illumination element, a water/air supply hole, a channel opening, and the like, but these components are not shown. An ultrasound probe 12 according to the embodiment is incorporated in the distal end part 10. The ultrasound probe 12 according to the embodiment may be incorporated in another endoscope. The ultrasound probe 12 may be used alone. A ground wiring technique and the like described later may be applied to other ultrasound probes (for example, an ultrasound probe that is in contact with a body surface).

In FIG. 1, an a-direction is a central axis direction of the ultrasound probe. Ab-direction is the longitudinal direction, which is the transducer arrangement direction. The b-direction intersects the a-direction. A direction intersecting the a-direction and the b-direction is the lateral direction. The a-direction is inclined with respect to an endoscope central axis (central axis of the distal end part 10). The a-direction can be referred to as an up-down direction as seen from the ultrasound probe 12. In this case, the up direction is an ultrasound emission direction.

The ultrasound probe 12 is a convex-type ultrasound probe. The ultrasound probe 12 has an assembly (second assembly, final assembly) 15, and the assembly 15 consists of the intermediate assembly (first assembly), a backing 14, and an acoustic lens 19. The intermediate assembly consists of a transducer array 16, a ground film 18, and a wiring sheet 26.

The transducer array 16 is composed of a plurality of transducers arranged in the longitudinal direction. In FIG. 1, an intermediate part of the transducer array 16 is not shown. Specifically, the transducer array 16 has an effective part 16A that transmits and receives ultrasound. The effective part 16A includes a plurality of real transducers 20 arranged in the longitudinal direction. In addition, the effective part 16A includes a plurality of first slits for separating the respective real transducers 20 from each other. The plurality of real transducers 20 and the plurality of first slits are alternately provided along the longitudinal direction.

The transducer array 16 has two dummy transducers 22 provided on both sides of the effective part 16A in the longitudinal direction. Each of the dummy transducers 22 does not exhibit an acoustic function, but exhibits an electrical function. As will be described in detail later, each of the dummy transducers 22 is a conductive member for the ground wiring. The transducer array 16 has two second slits (gaps) 24 provided between the two dummy transducers 22 and the effective part 16A.

The plurality of transducers of the transducer array 16 have the plurality of piezoelectric layers. The ground film 18 is provided on the upper side of the plurality of piezoelectric layers (however, the ground film 18 is not shown in FIG. 1). The ground film 18 is formed of, for example, a copper foil. In the embodiment, the upper surface electrodes (ground electrodes) of the plurality of piezoelectric layers of the plurality of transducers are bonded to the ground film 18.

The acoustic lens 19 is provided on the upper side of the transducer array 16. The acoustic lens 19 has a function of focusing the ultrasound in the lateral direction. The ground film 18 may be provided between the transducer array 16 and the acoustic lens 19. In this case, a conductive matching layer is provided as the matching layer in each transducer.

The wiring sheet 26 is provided on the lower side of the transducer array 16. The wiring sheet 26 is formed of a flexible printed circuit (FPC) substrate. In the embodiment, the wiring sheet 26 is formed of a multilayer FPC substrate. The wiring sheet 26 has a sheet body 28 provided on the lower side of the transducer array 16, a stretching part 30 pulled out from one end of the sheet body 28 in the longitudinal direction, and a stretching part 32 pulled out from the other end of the sheet body 28 in the longitudinal direction.

The sheet body 28 is bonded to an upper surface of the backing 14. The upper surface is a curved convex surface. The backing 14 is a member that attenuates the ultrasound emitted toward the lower side of the transducer array 16. The backing 14 has a first end part 14A and a second end part 14B that are separated from each other in the longitudinal direction. The first end part 14A has a first side surface intersecting the longitudinal direction. The second end part 14B has a second side surface intersecting the longitudinal direction. The backing 14 further has a third side surface and a fourth side surface intersecting with the lateral direction, and has a lower surface. The lower surface faces a lower space 33.

The stretching part 30 enters the lower space 33 through the space adjacent to the first end part 14A, and then makes a U-shaped turn in the lower space 33. The stretching part 30 has a first part 30a, a second part 30b, and a third part 30c. The first part 30a is a part that is in contact with the first side surface of the first end part 14A. The second part 30b is a part that is in contact with the lower surface of the backing 14. The third part 30c is a part that runs parallel to the second part 30b on the lower side of the second part 30b. A space between the sheet body 28 and the first part 30a is a bent part, a space between the first part 30a and the second part 30b is a bent part, and a space between the second part 30b and the third part 30c is a turn part.

The stretching part 32 enters the lower space 33 through a space adjacent to the second end part 14B. The stretching part 32 has a first part 32a and a second part 32b. The first part 32a is a part that is in contact with the second side surface of the second end part 14B. The second part 32b is a part that has entered a lower side of the third part 30c. A space between the sheet body 28 and the first part 32a is a bent part, and a space between the first part 32a and the second part 32b is a curved part.

A first connection region is provided on an upward surface of an end part of the third part 30c. A plurality of cables constituting a cable group 34 are connected to the first connection region. A second connection region is provided on a downward surface of an end part of the second part 32b. A plurality of cables constituting a cable group 36 are connected to the second connection region. Each of the cable groups 34 and 36 is composed of several tens or hundreds of cables. In FIG. 1, the cable groups 34 and 36 are schematically expressed. The end part of the third part 30c and the end part of the second part 32b overlap each other in the lower space 33. A length of the stretching part 30 and a length of the stretching part 32 are determined such that the overlapping occurs.

The intermediate assembly is composed of the wiring sheet 26, the transducer array 16, and the ground film 18. The intermediate assembly, the backing 14, and the acoustic lens 19 constitute the assembly (second assembly, final assembly) 15. The assembly 15 is disposed in the case of the distal end part 10. In a state in which the assembly 15 is disposed in the case, the lower space 33 is generated on the lower side of the backing 14. Two end parts of the wiring sheet 26 are accommodated in the lower space 33.

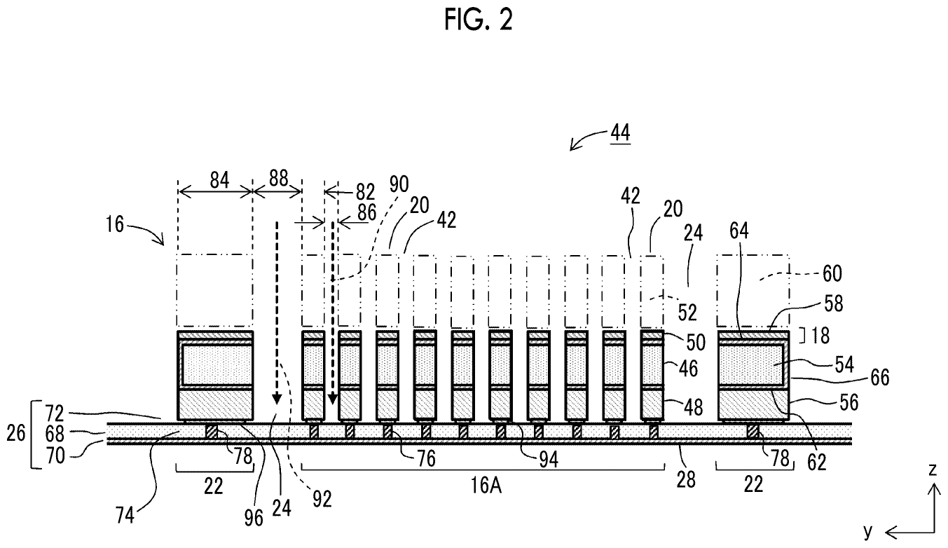

In FIG. 2, a vertical cross section of an intermediate assembly 44 is schematically shown. The intermediate assembly 44 is composed of the wiring sheet 26, the transducer array 16, and the ground film 18. In FIG. 2, the intermediate assembly 44 is in an unfolded state, that is, in a state before being curved. It should be noted that a y-direction, which is a horizontal direction, corresponds to the longitudinal direction, and a z-direction, which is a vertical direction, corresponds to the central axis direction.

The transducer array 16 has the effective part 16A, the two dummy transducers 22, and the two second slits 24. The effective part 16A has the plurality of real transducers 20 and a plurality of first slits 42. Each real transducer 20 has a piezoelectric layer (real piezoelectric layer) 46, a reflective layer (real reflective layer) 48, and a matching layer (real matching layer) 52. The matching layer 52 may be composed of a plurality of layers.

Each piezoelectric layer 46 has the signal electrode as the lower surface electrode and the ground electrode as the upper surface electrode. A strip (elongated piece) 50 in the ground film is bonded to the ground electrode. Stated another way, the strip 50 is interposed between the ground electrode and the matching layer 52.

The reflective layer 48 has a function of reflecting the ultrasound emitted from the piezoelectric layer 46 toward the lower side. An acoustic impedance of the reflective layer 48 is larger than an acoustic impedance of the piezoelectric layer 46. The piezoelectric layer 46 and the reflective layer 48 are integrated to form a resonator.

Each of the dummy transducers 22 has a dummy piezoelectric layer 54, a dummy reflective layer 56, and a dummy matching layer 60. The dummy piezoelectric layer 54 has the conductivity or the short-circuit function. Specifically, the dummy piezoelectric layer 54 has a lower surface electrode 62, an upper surface electrode 64, and a side surface electrode 66. The lower surface electrode 62 and the upper surface electrode 64 are electrically connected to each other through the side surface electrode 66, that is, the lower surface electrode 62 and the upper surface electrode 64 are short-circuited. A strip 58 is bonded to the upper surface electrode 64.

The plurality of piezoelectric layers 46 and the two dummy piezoelectric layers 54 are formed of the same piezoelectric material (for example, PZT). Each of a plurality of reflective layers 48 and two dummy reflective layers 56 is formed of the same conductive material. Each of the plurality of matching layers 52 and the two dummy matching layers 60 is formed of the same material. Each of the dummy transducers 22 does not function acoustically, but functions electrically. That is, the ground wiring line functions as an electrical component for the ground wiring.

The wiring sheet 26 has an insulating layer 68, a lower wiring pattern (lower wiring layer) 70, and an upper wiring pattern (upper wiring layer) 72. The sheet body 28 of the wiring sheet 26 has a via group 74. A plurality of signal vias 76 and a plurality of ground vias 78 are included in the via group 74. Each via is an element that electrically connects the lower electrode pattern and the upper electrode pattern, and specifically, is a through-hole having the conductivity.

The lower wiring pattern 70 includes a plurality of lines arranged in the lateral direction, and specifically, includes the plurality of lower signal lines and the plurality of lower ground lines. The upper wiring pattern 72 includes a plurality of lines arranged in the longitudinal direction, and specifically, includes the plurality of upper signal lines and the plurality of upper ground lines. Each line corresponds to an electrode or an electrical path.

The plurality of lower signal lines are connected to a plurality of upper signal lines 94 through the plurality of signal vias 76. The plurality of upper signal lines 94 are connected to the plurality of signal electrodes of the plurality of piezoelectric layers 46 through the plurality of reflective layers 48.

In each of the dummy transducers 22, the dummy piezoelectric layer 54 has the conductivity, and the dummy reflective layer 56 on the lower side thereof also has the conductivity. Therefore, in each of the dummy transducers 22, the dummy piezoelectric layer 54 and the dummy reflective layer 56 constitute the conductive part. For example, the lower ground line is connected to the upper ground line 96 through the ground via 78. The upper ground line 96 is connected to the ground film 18 through the conductive part.

The lower ground line and the upper ground line may be connected to each other through the plurality of ground vias. A structure in which the ground line and the ground film are connected to each other may be provided in the effective part 16A.

For example, in each piezoelectric layer 46, a length in the lateral direction is, for example, 2 mm or 3 mm, a width 82 in the y-direction is, for example, 100 μm, and a thickness in the z-direction is, for example, 100 μm. In each dummy piezoelectric layer 54, a length in the lateral direction is, for example, 2 mm or 3 mm, a width 84 in the y-direction is, for example, 150 μm, and a thickness in the z-direction is, for example, 100 μm. The numerical values described in the present specification are merely examples.

In the embodiment, the width 84 is larger than the width 82. As a result, the electric resistance of the ground path is reduced. The structures of both end parts of the transducer array 16 are physically reinforced. The collapse of each of the dummy transducers 22 is unlikely to occur. The two dummy transducers 22 are provided on both sides of the effective part 16A in the longitudinal direction, so that the effective part 16A is physically protected.

A width 86 of each of the first slits 42 in the y-direction is, for example, 50 μm. A width 88 of each second slit 24 in the y-direction is, for example, 100 μm. The width 88 is larger than the width 86. As a result, it is possible to effectively prevent an electrical short-circuit between the real transducers at both end parts of the effective part 16A and the two dummy transducers 22 adjacent thereto. For example, in the repolarization processing of the effective part 16A, a high voltage is applied to the effective part 16A. In this case, the short-circuit or polarization failure can be prevented.

In each piezoelectric layer 46, the lower surface electrode (signal electrode) and the upper surface electrode (ground electrode) are formed by gold plating or the like. The thicknesses thereof are, for example, 0.3 μm. In each dummy piezoelectric layer 54, the lower surface electrode 62, the upper surface electrode 64, and the side surface electrode 66 are formed by gold plating or the like. The thicknesses thereof are, for example, 0.3 μm. The ground film 18 includes, for example, a copper foil having a thickness of 5 μm. A surface of the ground film 18 is also subjected to gold plating. A thickness of a gold plating layer is, for example, 0.3 μm.

In a manufacturing process of the ultrasound probe, as will be described later, a reflective material plate, a piezoelectric material plate, and a matching material plate are laminated on the sheet body 28 of the wiring sheet 26. These components are bonded to each other. As a result, a laminate is formed. In addition, dicing 90 and dicing 92 using a dicing saw is performed on the laminate. As a result, the intermediate assembly 44 is formed. The intermediate assembly 44 is bonded to the upper surface of the backing. In addition, the acoustic lens is bonded to the upper side of the intermediate assembly 44. The final assembly is produced through the above-described process. The final assembly corresponds to an ultrasound sensor. The final assembly is disposed in the case.

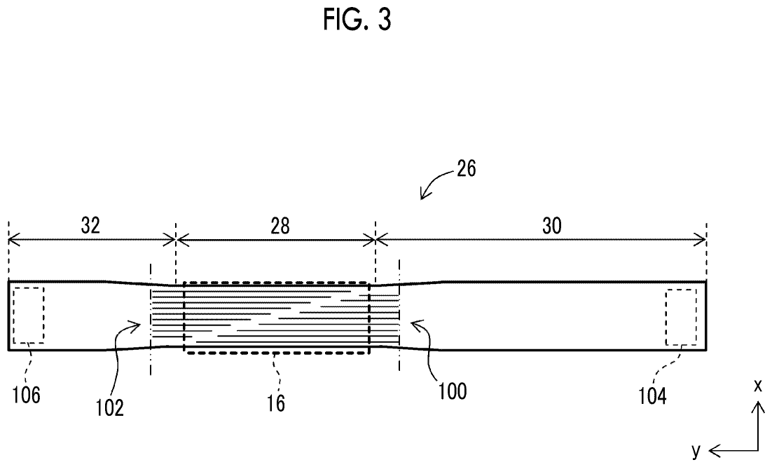

FIG. 3 shows the wiring sheet 26 in the unfolded state. The wiring sheet 26 is composed of the sheet body 28, the stretching part 30, and a stretching part 23. The y-direction is the longitudinal direction, and an x-direction is the lateral direction. The transducer array 16 is provided on the sheet body 28. The wiring sheet 26 has the lower wiring pattern. A first lower wiring pattern 100 and a second lower wiring pattern 102 are included in the lower wiring pattern.

The first lower wiring pattern 100 is formed over the sheet body 28 and the stretching part 30. In FIG. 3, a part of the first lower wiring pattern 100 is not shown. The second lower wiring pattern 102 is formed over the sheet body 28 and the stretching part 32. In FIG. 3, a part of the second lower wiring pattern 102 is not shown. A connection region 104 is provided at an end part of the stretching part 30. A connection region 106 is provided at an end part of the stretching part 32.

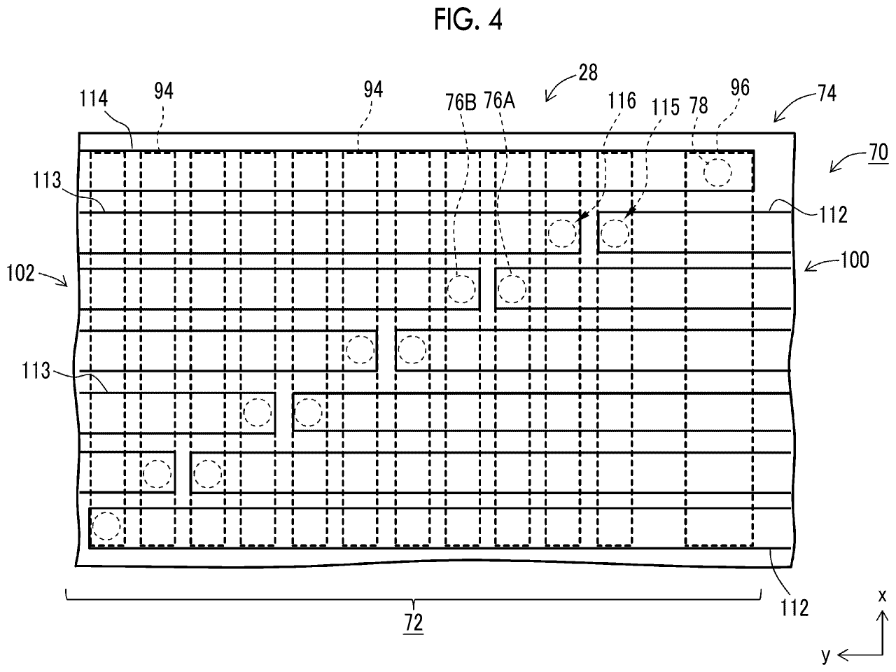

FIG. 4 shows a part of the sheet body 28. The upper wiring pattern 72 is composed of the plurality of lines arranged in the longitudinal direction. Specifically, the upper wiring pattern 72 is composed of the plurality of upper signal lines 94 and two upper ground lines 96. However, in FIG. 4, only one upper ground line 96 among the two upper ground lines 96 is shown.

As described above, the lower wiring pattern 70 is composed of the first lower wiring pattern 100 and the second lower wiring pattern 102. Specifically, the first lower wiring pattern 100 is composed of the first lower signal line group and a first lower ground line, and the second lower wiring pattern 102 is composed of the second lower signal line group and a second lower ground line. Hereinafter, the wiring pattern in the wiring sheet will be described in more detail.

The plurality of real transducers are composed of n real transducers from the first real transducer to the n-th real transducer that are arranged in order of numbers in the longitudinal direction (n is an integer of 2 or more, and is, for example, 60 or more and 300 or less). The n real transducers are connected to n upper signal lines 94. The two dummy transducers are connected to the two upper ground lines 96.

The first lower wiring pattern 100 includes a plurality of first lower signal lines 112 connected to the odd-numbered real transducers and includes the first lower ground line connected to one dummy transducer. The first lower signal line group is composed of the plurality of first lower signal lines 112 connected to the odd-numbered real transducers.

The second lower wiring pattern 102 includes a plurality of second lower signal lines 113 connected to even-numbered real transducers and includes a second lower ground line 114 connected to the other dummy transducer. A second lower signal line group is composed of the plurality of second lower signal lines 113 connected to the even-numbered real transducers.

The via group 74 includes a first via row 115 and a second via row 116, and also includes the plurality of ground vias 78. The first via row 115 is composed of a plurality of signal vias 76A connected to the odd-numbered real transducers, and the second via row 116 is composed of a plurality of signal vias 76B connected to the even-numbered real transducers. The first via row 115 is formed along a diagonal direction of the sheet body 28, and the second via row 116 is also formed along the diagonal direction of the sheet body 28. However, the first via row 115 and the second via row 116 are in a relationship in which the first via row 115 and the second via row 116 are shifted from each other in the y-direction.

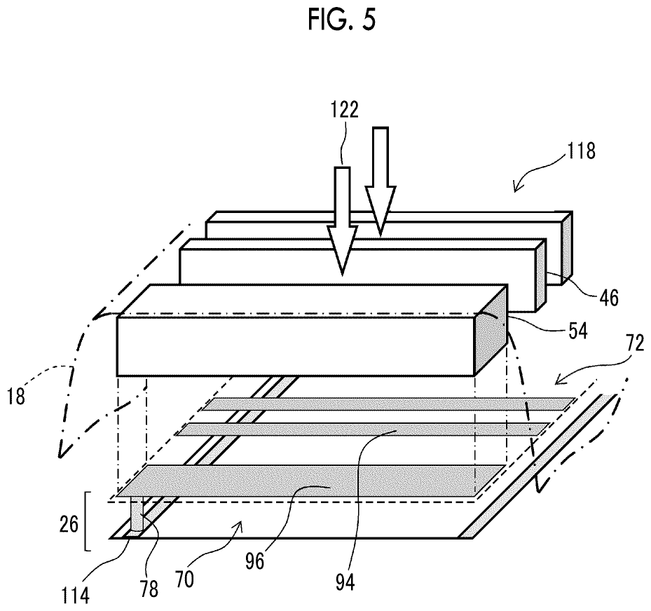

FIG. 5 shows a piezoelectric layer array 118 and the wiring sheet 26. The piezoelectric layer array 118 is produced by dicing 122 and is composed of the plurality of piezoelectric layers arranged in the longitudinal direction. Specifically, the piezoelectric layer array 118 includes the plurality of piezoelectric layers (real piezoelectric layers) 46 and the two dummy piezoelectric layers 54. The ground film 18 is provided on the upper side of the piezoelectric layer array 118. However, in FIG. 5, the ground film 18 is schematically expressed.

The wiring sheet 26 includes the lower wiring pattern 70 and the upper wiring pattern 72. In FIG. 5, the ground line (second lower ground line) 114 in the lower wiring pattern 70 is shown. The ground line 114 is connected to the upper ground line 96 through the ground via 78. The upper ground line 96 is connected to the ground film 18 through the dummy reflective layer (not shown) and the dummy piezoelectric layer 54.

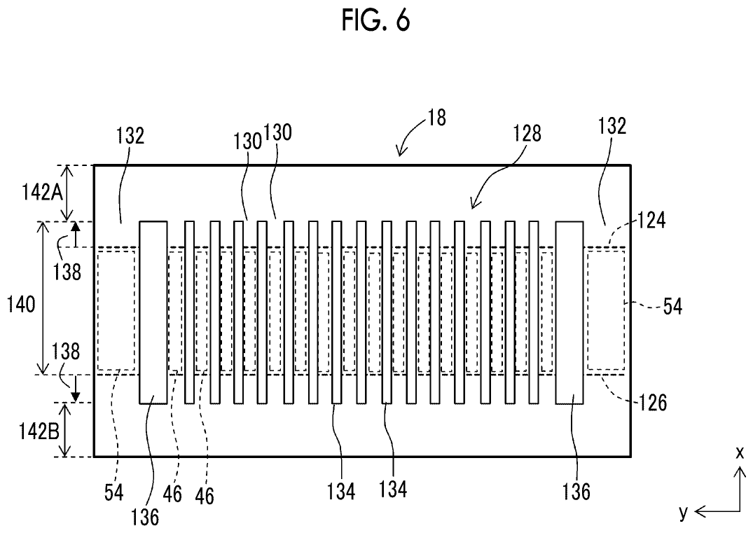

FIG. 6 shows the ground film 18 in through unfolded state. The ground film 18 is easily deformed. The ground film 18 is composed of a film body 140 and a pair of film end parts 142A and 142B connected to both sides of the film body 140 in the lateral direction. Reference numerals 124 and 126 each denote a folding line.

The film body 140 has a strip row 128 consisting of the plurality of strips arranged in the longitudinal direction. The strip row 128 includes a plurality of strips 130 bonded to the upper surface electrodes of the plurality of piezoelectric layers (real piezoelectric layers) 46 and two strips 132 bonded to the upper surface electrodes of the two dummy piezoelectric layers 54.

The film body 140 has a plurality of openings 134 corresponding to the plurality of first slits and two openings 136 corresponding to the two second slits. The plurality of openings 134 and 136 are formed by performing dicing on the laminate. Reference numeral 138 denotes a dicing depth. In a case of producing the assembly, the two film end parts 142A and 142B may be attached to the two side surfaces of the backing.

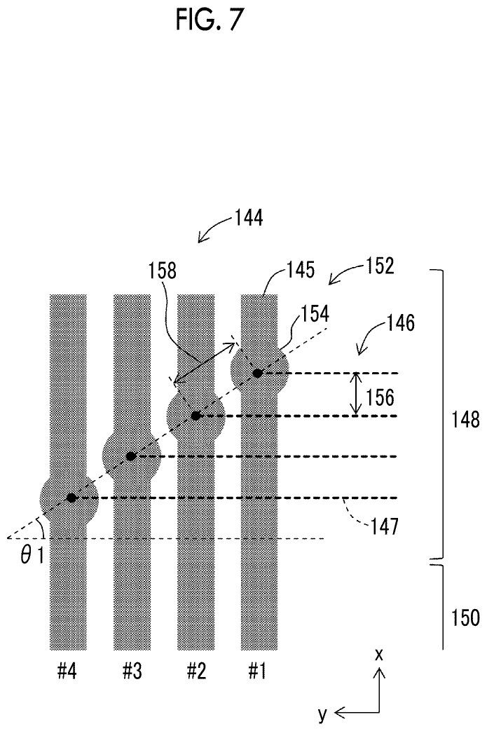

FIGS. 7 to 9 show several wiring patterns. In each drawing, #1 denotes a first upper signal line connected to the first transducer (real transducer), and #2 denotes a second upper signal line connected to the second transducer (real transducer). The same applies to #3, #4, and the like.

In FIG. 7, a first example of the wiring pattern is shown. An upper wiring pattern 144 includes a plurality of upper signal lines 145 arranged in the longitudinal direction (y-direction). A lower wiring pattern 146 is divided into a first part 148 and a second part 150 arranged in the lateral direction (x-direction). The first part 148 includes a plurality of lower signal lines 147 pulled out to a right side in FIG. 7. The second part 150 includes a plurality of lower signal lines pulled out to a left side in FIG. 7. A via group 152 is composed of a plurality of vias 154 arranged in the diagonal direction of the sheet body.

In FIG. 7, reference numeral 158 denotes an inter-via pitch. 01 denotes an inclination angle of the via group 152.

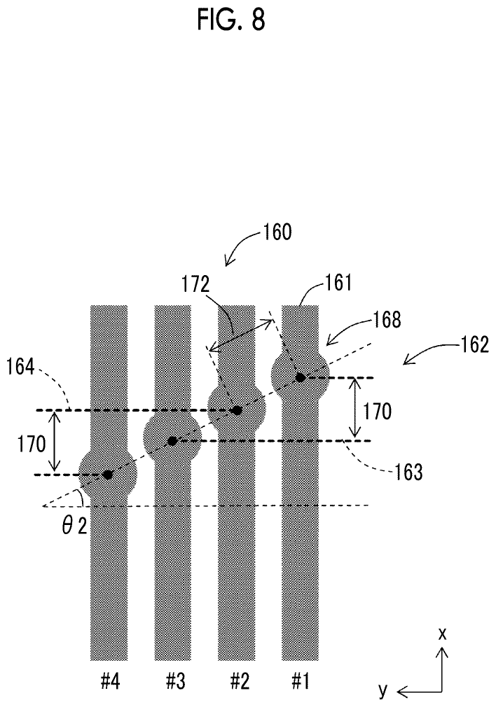

In FIG. 8, a second example of the wiring pattern is shown. An upper wiring pattern 160 includes a plurality of upper signal lines 161 arranged in the longitudinal direction (y-direction). A lower wiring pattern 162 includes a plurality of lower signal lines arranged in the lateral direction. More specifically, the plurality of lower signal lines consist of odd-numbered lower signal lines 163 and even-numbered lower signal lines 164. These signal lines are alternately disposed in the lateral direction. The via group 168 is composed of a plurality of vias arranged in the diagonal direction of the sheet body.

In FIG. 8, reference numeral 172 denotes an inter-via pitch. 02 denotes an inclination angle of the via group 152. According to the second example shown in FIG. 8, the inter-via pitch 172 can be increased as compared with the first example, that is, the inclination angle θ2 can be reduced. Therefore, according to the second example, a width of the wiring sheet in the x-direction can be reduced.

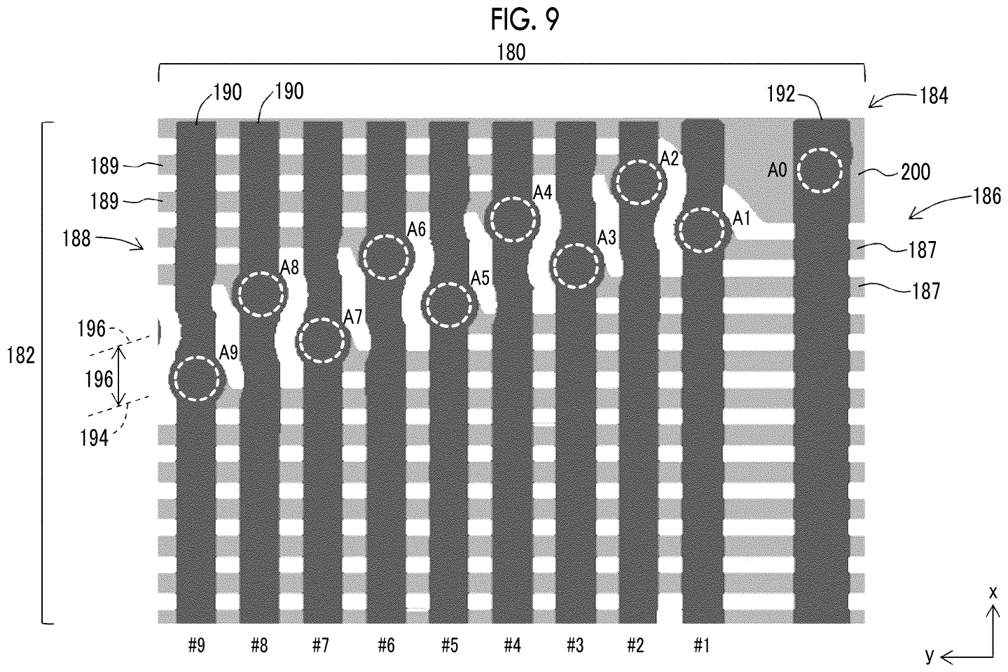

In FIG. 9, a third example of the wiring pattern is shown. An upper wiring pattern 180 includes a plurality of upper signal lines 190 and two upper ground lines 192 arranged in the longitudinal direction. However, in FIG. 9, only one upper ground line among the two upper ground lines 192 is shown.

A lower wiring pattern 182 includes a first lower wiring pattern 186 and a second lower wiring pattern 188. The first lower wiring pattern 186 includes a plurality of lower signal lines 187 and a lower ground line. The second lower wiring pattern 188 includes a plurality of lower signal lines 189 and a lower ground line 200.

More specifically, the lower wiring pattern 182 includes n lower signal lines from a first lower signal line to an n-th lower signal line. The n lower signal lines are connected to the n transducers (real transducers). The odd-numbered lower signal lines 187 are connected to the odd-numbered transducers (real transducers). The even-numbered lower signal lines 189 are connected to the even-numbered transducers (real transducers). The odd-numbered lower signal lines 187 and the even-numbered lower signal lines are alternately disposed in the lateral direction.

The via group 184 is composed of a first via row 194 and a second via row 196. The first via row is composed of odd-numbered first signal vias A1, A3, A5, A7, A9, and the like that are connected to the odd-numbered lower signal lines 187. The second via row 196 is composed of even-numbered second signal vias A2, A4, A6, A8, and the like that are connected to the even-numbered lower signal lines 189.

The first via row 194 and the second via row 196 are formed along the diagonal direction of the sheet body. The first via row 194 and the second via row 196 are shifted in the y-direction (see reference numeral 196). It can also be said that these via rows are shifted in the x-direction. The n signal vias are disposed in a zigzag pattern from the first signal via to the n-th signal via. It should be noted that A0 denotes a ground via. One or a plurality of lower ground lines may be connected to one upper ground line 192 through the plurality of ground vias.

In a case in which the third example is adopted, the inter-via pitch can be further increased as compared with the second example, that is, the inclination angles of the first via row 194 and the second via row 196 can be further reduced. Therefore, according to the third example, the width of the wiring sheet in the x-direction can be further reduced. Further, according to the third example, it is also easy to reduce the inter-transducer pitch.

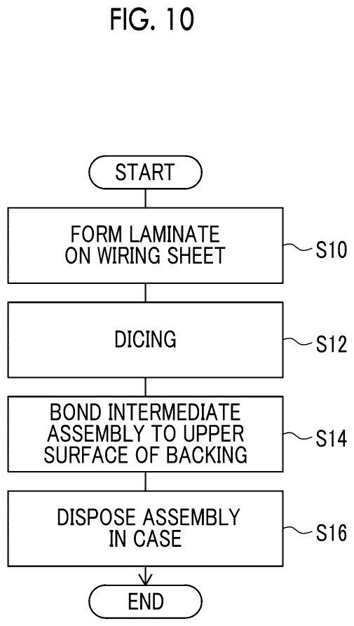

FIG. 10 shows a method of manufacturing the ultrasound probe according to the embodiment. In S10, the reflective material plate, the piezoelectric material plate, the ground film, and one or a plurality of the matching material plates are laminated on the sheet body in the wiring sheet, and these components are bonded to each other. As a result, a laminate is produced on the wiring sheet.

In S12, the dicing is performed on the laminate. As a result, the transducer array is produced from the laminate. The intermediate assembly is composed of the wiring sheet and the transducer array. In S14, the intermediate assembly is bonded to the upper surface of the backing. Further, the acoustic lens is bonded to the upper side of the transducer array. As a result, the assembly is produced. The cable group is connected to the two stretching parts in the assembly. In S16, the assembly is disposed in the case. As a result, the ultrasound probe shown in FIG. 1 is formed.

According to the above-described embodiment, it is possible to easily and reliably perform the ground wiring by using the dummy transducer. Therefore, it is possible to achieve a transducer array having good acoustic characteristics. In the above-described embodiment, in the transducer array, since the two dummy transducers are provided on both sides of the effective part in the longitudinal direction, the physical strength of the transducer array can be increased. In particular, an advantage of preventing the occurrence of the transducer collapse at both end parts of the transducer array can be obtained. Further, in the above-described embodiment, in the wiring sheet, the odd-numbered lower signal lines and the even-numbered lower signal lines are alternately disposed, so that electrical crosstalk in the wiring sheet can be reduced. The feature matters regarding the wiring pattern in the wiring sheet can also be adopted in the ultrasound probe that does not have the dummy transducer.

What is claimed is:

1. An ultrasound probe comprising:
a transducer array that has a plurality of real transducers having a plurality of real piezoelectric layers and a dummy transducer having a dummy piezoelectric layer;
a ground film that is provided on an upper side of the plurality of real piezoelectric layers and the dummy piezoelectric layer and that is electrically connected to a plurality of ground electrodes of the plurality of real piezoelectric layers and an upper surface electrode of the dummy piezoelectric layer; and
a wiring sheet that is provided on a lower side of the transducer array and that has a plurality of signal lines electrically connected to a plurality of signal electrodes of the plurality of real piezoelectric layers and a ground line electrically connected to the ground film,
wherein the dummy transducer has a conductive part that is provided between the ground film and the wiring sheet and that includes the dummy piezoelectric layer, and
the ground film and the ground line are electrically connected to each other through the conductive part.

2. The ultrasound probe according to claim 1,
wherein the dummy piezoelectric layer has the upper surface electrode, a lower surface electrode, and a side surface electrode connected to the upper surface electrode and the lower surface electrode.

3. The ultrasound probe according to claim 1,
wherein the plurality of real transducers have a plurality of real reflective layers that are provided on a lower side of the plurality of real piezoelectric layers and that have conductivity, and
the conductive part has a dummy reflective layer that is provided on a lower side of the dummy transducer and that has conductivity.

4. The ultrasound probe according to claim 1,
wherein the transducer array has a longitudinal direction, and
a width of the dummy transducer in the longitudinal direction is larger than a width of each real transducer in the longitudinal direction.

5. The ultrasound probe according to claim 1,
wherein the transducer array includes
an effective part that is composed of the plurality of real transducers and a plurality of first slits for separating the respective real transducers from each other, and
a second slit that is provided between the effective part and the dummy transducer,
the transducer array has a longitudinal direction, and
a width of the second slit in the longitudinal direction is larger than a width of each of the first slits in the longitudinal direction.

6. The ultrasound probe according to claim 1,
wherein the ground film includes
a film body that has a plurality of strips electrically bonded to the plurality of ground electrodes and the upper surface electrode, and
a pair of film end parts that are connected to the plurality of strips.

7. The ultrasound probe according to claim 1,
wherein the transducer array has a longitudinal direction and a lateral direction intersecting the longitudinal direction, and
the wiring sheet includes
an upper wiring pattern having a plurality of upper signal lines that are electrically connected to a plurality of signal electrodes of the plurality of real transducers and that are arranged in the longitudinal direction and an upper ground line that is electrically connected to a lower surface electrode of the dummy transducer,
a lower wiring pattern having a plurality of lower signal lines that are arranged in the lateral direction and a lower ground line, and
a via group having a plurality of signal vias that connect the plurality of upper signal lines and the plurality of lower signal lines to each other and a ground via that electrically connects the upper ground line and the lower ground line to each other.

8. The ultrasound probe according to claim 7,
wherein the wiring sheet includes
a sheet body that is provided on the lower side of the transducer array and that has one end and the other end which are separated from each other in the longitudinal direction,
a first stretching part that is pulled out from the one end of the sheet body, and
a second stretching part that is pulled out from the other end of the sheet body,
the plurality of real transducers are n real transducers from a first real transducer to an n-th real transducer that are arranged in order of numbers in the longitudinal direction, and
the plurality of lower signal lines include
a first lower signal line group that is electrically connected to odd-numbered real transducers among the n real transducers and that is provided over the sheet body and the first stretching part, and
a second lower signal line group that is electrically connected to even-numbered real transducers among the n real transducers and that is provided over the sheet body and the second stretching part.

9. The ultrasound probe according to claim 8,
wherein the plurality of signal vias include
a first signal via row that is connected to the first lower signal line group, and
a second signal via row that is connected to the second lower signal line group,
the first signal via row is aligned along a diagonal direction of the sheet body, and
the second signal via row is aligned along the diagonal direction while being shifted from the first signal via row.

10. The ultrasound probe according to claim 1, further comprising:
a first assembly that consists of the transducer array, the ground film, and the wiring sheet;
a backing that is provided on a lower side of the first assembly and that has a first end part and a second end part which are separated from each other in a longitudinal direction; and
a case that accommodates a second assembly consisting of the first assembly and the backing,
wherein the wiring sheet includes
a sheet body that is provided on the lower side of the transducer array and that has one end and the other end which are separated from each other in the longitudinal direction,
a first stretching part that is pulled out from the one end of the sheet body, and
a second stretching part that is pulled out from the other end of the sheet body, a lower space is formed on a lower side of the backing in the case,
the first stretching part enters the lower space through a space adjacent to the first end part and then makes a U-shaped turn in the lower space,
the second stretching part enters the lower space through a space adjacent to the second end part, and
an end part of the first stretching part and an end part of the second stretching part overlap each other in the lower space.
